(19) United States
(12) Patent Application Publication
(10) Pub. No.: US 2022/0214357 A1
(43) Pub. Date: Jul. 7, 2022

(54) METHOD OF DETERMINING THE PROBABILITY OF INFLAMMATORY BOWEL DISEASE IN A SUBJECT BEING ULCERATIVE COLITIS OR CROHN'S DISEASE

(71) Applicant: University of Limerick, Limerick (IE)

(72) Inventors: Colum DUNNE, Co. Tipperary (IE); J. Calvin COFFEY, Limerick (IE); Miranda KIERNAN, Co. Cavan (IE)

(73) Assignee: University of Limerick, Limerick (IE)

(21) Appl. No.: 17/607,707

(22) PCT Filed: May 4, 2020
(86) PCT No.: PCT/EP2020/062354

§ 371 Date: Oct. 29, 2021

(30) Foreign Application Priority Data
May 2, 2019 (EP) 19172409.5

(51) Int. Cl. G01N 33/68; G01N 33/50

(52) U.S. Cl. CPC G01N 33/6893; G01N 33/5023; G01N 2800/065; G01N 2333/5428; G01N 2333/495; G01N 33/6869

(57) ABSTRACT

The Applicant has discovered a cytokine profile that can accurately distinguish between ulcerative colitis (UC) and Crohn's disease (CD) in a subject having, or having symptoms of, inflammatory bowel disease (IBD). The profile of ulcerative colitis is increased IL-10 levels (compared with a reference IL-10 level, and decreased IL-23 levels (compared with a reference IL-23 level). The cytokine profile can be detected at a protein or genomic level, and is generally determined from a peripheral blood sample (i.e. a blood fraction such as serum, plasma, or blood cells such as peripheral blood mononuclear cells). Distinguishing between UC and CD in a subject with IBD allows a clinician prescribe a suitable therapeutic regime for the subject using a non-invasive blood test, and avoiding the need for scoping or tissue biopsy, which are undesirable for the subject. The cytokine profile can also be used to monitor a therapeutic regime for effectiveness.

### FIELD OF THE INVENTION

The present invention relates to a method of determining the probability of inflammatory bowel disease in a subject being ulcerative colitis or Crohn's disease. Also contemplated are methods of monitoring treatment of ulcerative colitis or Crohn's disease.

### BACKGROUND TO THE INVENTION

Inflammatory bowel diseases (IBD) are chronic, relapsing inflammatory conditions that are immunologically mediated. They include both Crohn's disease (CD) and ulcerative colitis (UC). In general, IBD is believed to result from an aggressive immunologic response in genetically susceptible individuals, usually due to an environmental factor, such as gut commensals. IBD can be diagnosed at any age, but the majority of diagnoses are made between the ages of 20 and 30, with a second peak of IBD diagnoses occurs during the sixth or seventh decade of life. Due to the early onset of IBD, the severe symptoms associated with it, the natural unsettled course of disease, number of hospitalisations and the lack of a cure, IBD diagnosis has a significant impact on a patient's quality of life. The highest prevalence rates of CD and UC are found in Europe, 322 and 505 cases per 100,000 persons (2.2 million people), followed by North America, with 319 and 249 cases per 100,000 persons (1.4 million people), respectively. In Ireland, there are between 1.95-3.76 and 3.10-4.97 per 100,000 population reported new diagnoses of CD and UC, respectively, per year. In summary, the incidence rate (the rate of new cases per population) of IBD is increasing, and at elevated levels in countries previously considered as low-incidence and low-prevalence areas such as South America, Africa and Asia, thought to be due to their transition to western-style lifestyles.

When IBD predominantly involves the colon, differentiation between CD and UC is often challenging. Inaccurate diagnoses are estimated to occur in 30% of IBD patients. In most cases the diagnostic uncertainty arises from the overlap of clinical and histologic features, making CD appear like UC. This scenario is particularly relevant to young children, a population in which IBD consists of up to 80%. The differentiation between UC and CD relies on a compilation of clinical, radiologic, endoscopic, and histopathologic interpretations; a compilation that is not always accurate.

An estimated 15% of IBD patients are indistinguishable following one or more of clinicial, radiologic, serologic and pathological tests and are labelled as “Indeterminate Colitis” (IC). Indeterminate Colitis is described in the literature, including:

Odze et al. (Modern Pathology 28, S30-S46, 2015); Guindi et al. (J. Clin. Pathol. 2004 December; 57(12): 1233-1244); and Telakis et al. (Annals of Gastorenterology 2008, 21(3): 173-179). In addition, another 15% of the colonic IBD cases that undergo pouch surgery resulting from a definitive UC diagnosis (based on the pathologist's initial designation of endoscopic biopsies and colectomy specimen) will have their original UC diagnosis changed to CD based on the postoperative follow-up when clinical and histopathology changes indicate development of CD in the ileal pouch. One-half of these patients will require pouch excision or diversion.

Due to the unpredictable nature of IBD, side effects of medications, and potential complications, some of which may end in sudden incapacitation, IBD is becoming a global health concern. Distinguishing between CD and UC is critical to therapy. The clinical experience suggests that identifying patients with Crohn's colitis and positive outcomes after pouch surgery is arduous. Thus, restorative proctocolectomy (RPC) should be contraindicated for CD patients, whereas ileal pouch-anal anastomosis (IPAA) is standard acceptable care for patients with UC and IC who are predicted likely to develop UC. Inevitably, pouch complications are significantly higher in patients with CD (±64%) and IC (±43%) vs patients having UC (±22%) (P<0.05).

This diagnostic dilemma and the potential morbidity from a wrong diagnosis and unnecessary and/or inappropriate surgical interventions underscore the importance of research strategy focused at improving diagnosis of the colitides using molecular biometrics.

To date, there has been significant interest in attempting to identify molecular biomarkers that can accurately delineate CD and UC phenotypes. These studies have been minimally successful at identifying such biomarkers. In serum these include: placenta growth factor-1 (PLGF-1), IL-7, TGFβ1, and IL-12P40. In biopsies obtained from the mucosa, they are Rho GD1α, desmoglein, pleckstrin, VDAC (voltage-dependent anion channel), 3-hydroxy-3-methylglutaryl-coenzyme A reductase (HMG-CoA), and C10orf76. In stool they are calprotectin, PMN-elastate, lactoferrin, and S100A12.

Clearly these biomarbiometrics represent an advance in the field of colitides research and have been used for clinical prognostic trials but have not been shown to delineate UC from a CD phenotype. This diagnostic dilemma and the potential morbidity from a wrong diagnosis and unnecessary and/or inappropriate surgical interventions underscore the importance of research strategy focused at improving diagnosis of the colitides using molecular biometrics.

Sakuraba et al. (Gastroenterology 2009; 137: 1736-1745) teaches that the mesenteric lymph node is the key pathogenic location of Crohn's Disease elicited by dendritic cell produced cytokines ultimately leading to a dysregulated TH1/Th17 immune response. The paper notes that when stimulated with exogenous bacterial derivative, myeloid dendritic cells from the mesenteric lymph node of CD subjects produced a higher amount of IL-23 and a lower amount of IL-10, and induced a stronger T helper cell immune response in mixed lymphocyte reaction compared with those from UC and a normal control.

It is an object of the invention to overcome at least one of the above-referenced problems.

### SUMMARY OF THE INVENTION

The Applicant has discovered a cytokine profile that can accurately distinguish between ulcerative colitis (UC) and Crohn's disease (CD) in a subject having, or having symptoms of, inflammatory bowel disease (IBD). The profile of ulcerative colitis is increased IL-10 levels (compared with a reference IL-10 level, and decreased IL-23 levels (compared with a reference IL-23 level). The cytokine profile can be detected at a protein or genomic level, and is generally determined from a peripheral blood sample (i.e. a blood fraction such as serum, plasma, or blood cells such as peripheral blood mononuclear cells). Distinguishing between UC and CD in a subject with IBD allows a clinician prescribe a suitable therapeutic regime for the subject using a non-invasive blood test, and avoiding the need for scoping or tissue biopsy, which are undesirable for the subject. The cytokine profile can also be used to monitor a therapeutic regime for effectiveness. The methods of the invention can be embodied with any suitable in-vitro diagnostics technique, including antibody-based techniques (i.e. immunoassays such as a quantitative ELISA), or nucleic acid-based techniques such as quantitative mRNA profiling. The method of the invention is particularly applicable to inflammatory bowel disease subjects that are diagnosed as indeterminate colitis (IC) subjects, where the method may be employed to identify the likely form of inflammatory bowel disease.

The invention broadly provides a method of determining inflammatory bowel disease (IBD) status in a subject (for example classifying the type of IBD, determining the probability of the disease being ulcerative colitis or Crohn's disease, distinguishing between ulcerative colitis or Crohn's disease, or detecting or predicting ulcerative colitis or Crohn's disease, in particular a subject as having Indeterminate Colitis (IC) The subject is generally symptomatic of IBD, as determined by usual methods (i.e. loss of weight, abdominal cramps, blood in stool) and the method of the invention is employed to classify the IBD prior to treatment of the disease. The patient may also have no symptoms, but may be genetically predisposed to the disease.

The method typically comprises the step of detecting increased or decreased levels of IL-10 and IL-23 (relative to reference control values from a healthy subject), and correlating the levels of IL-10 and IL-23 with type of IBD.

In one embodiment, an increased level of IL-10 compared with the reference level of IL-10 and a decreased level of IL-23 compared with the reference level of II-23 is indicative of a probability of the inflammatory bowel disease being ulcerative colitis as opposed to Crohn's disease.

In another embodiment, an increased level of IL-23 compared with the reference level of IL-23 and a decreased level of II-10 compared with the reference level of IL-10 is indicative of a probability of the inflammatory bowel disease being Crohn's disease as opposed to ulcerative colitis.

The methods of the invention may also include the step of detecting an increase or decrease in levels of additional cytokines (relative to reference control values), for example TGFß-1 and/or TGFß-2, where a decreased level of TGFß-1 or increased level of TGFß-1 (compared with a reference control levels) is indicative of a probability of the inflammatory bowel disease being ulcerative colitis as opposed to Crohn's disease, and where an increased level of TGFß-1 or decreased level of TGFß-1 (compared with a reference control levels) is indicative of a probability of the inflammatory bowel disease being Crohn's disease as opposed to ulcerative colitis.

In one embodiment, the method comprises the steps of:
determining a level of a plurality of biomarkers in a blood sample obtained from the subject with inflammatory bowel disease, in which the biomarkers include IL-10 and IL-23; wherein
   an increased level of IL-10 compared with a reference control level of IL-10 and a decreased level of IL-23 compared with a reference control level of IL-23 is indicative of a probability of the inflammatory bowel disease being ulcerative colitis as opposed to Crohn's disease; and
   an increased level of IL-23 compared with the reference control level of IL-23 and a decreased level of IL-10 compared with the reference control level of IL-10 is indicative of a probability of the inflammatory bowel disease being Crohn's disease as opposed to ulcerative colitis.

In one embodiment, the method comprises the steps of comparing the measured level of IL-10 with a reference level of IL-10 and comparing the measured level of IL-23 with a reference level of IL-23.

Typically, the plurality of blood-borne biomarkers consist of cytokines.

In one embodiment, the plurality of biomarkers consist of (a) IL-10, (b) IL-23, and (c) TGFß-1 and/or TGFß-2.

In one embodiment, which the plurality of biomarkers consist of IL-10 and IL-23.

In one embodiment, the subject has been diagnosed with indeterminate colitis. Thus, the method may be employed to determine whether the IBD in the subject identified as having indeterminate colitis (IC) is likely to be UC or CD.

In one embodiment, the subject has been diagnosed with indeterminate colitis by diagnostic analysis typically comprising a method selected from the group consisting of: scoping of the digestive tract; biopsy; MRI; CT scan; or ultrasound.

In one embodiment, the method includes a step of determining a level of TGFß-1 in a sample obtained from the subject and comparing the measured level of TGFß-1 with a reference level of TGFß-1 from a healthy subject, wherein:

an increased level of IL-10 compared with the reference level of IL-10, a decreased level of IL-23 compared with the reference level of IL-23, and a decreased level of TGFß-1 compared with a reference TGFß-1 level, is indicative of a probability of the inflammatory bowel disease being ulcerative colitis as opposed to Crohn's disease.

In one embodiment, the method includes a step of determining a level of TGFß-1 in a sample obtained from the subject and comparing the measured level of TGFß-1 with a reference level of TGFß-1 from a healthy subject, wherein:

an increased level of IL-23 compared with the reference level of IL-23, a decreased level of IL-10 compared with the reference level of IL-10, and an increased level of TGFß-1 compared with a reference TGFß-1 level, is indicative of a probability of the inflammatory bowel disease being Crohn's disease as opposed to ulcerative colitis.

In one embodiment, the method includes a step of determining a level of TGFß-2 in a sample obtained from the subject and comparing the measured level of TGFß-2 with a reference level of TGFß-2 from a healthy subject, wherein:

an increased level of IL-10 compared with the reference level of IL-10, a decreased level of IL-23 compared with the reference level of IL-23, and an increased level of TGFß-2 compared with a reference TGFß-2 level, is indicative of a probability of the inflammatory bowel disease being ulcerative colitis as opposed to Crohn's disease.

In one embodiment, the method includes a step of determining a level of TGFß-2 in a sample obtained from the subject and comparing the measured level of TGFß-2 with a reference level of TGFß-2 from a healthy subject, wherein:

an increased level of IL-23 compared with the reference level of IL-23, a decreased level of IL-10 compared with the reference level of IL-10, and a decreased level of TGFß-2 compared with a reference TGFß-2 level, is indicative of a probability of the inflammatory bowel disease being Crohn's disease as opposed to ulcerative colitis.

In one embodiment, the method includes the steps of determining a level of TGFß-1 in a sample obtained from the subject and comparing the measured level of TGFß-1 with a reference level of TGFß-1 from a healthy subject, and determining a level of TGFß-2 in a sample obtained from the subject and comparing the measured level of TGFß-2 with a reference level of TGFß-2 from a healthy subject, wherein:

an increased level of IL-10 compared with the reference level of IL-10, a decreased level of IL-23 compared with the reference level of IL-23, a decreased level of TGFß-1 compared with a reference TGFß-1 level, and an increased level of TGFß-2 compared with a reference level of TGFß-2, is indicative of a probability of the inflammatory bowel disease being ulcerative colitis as opposed to Crohn's disease.

In one embodiment, the method includes the steps of determining a level of TGFß-1 in a sample obtained from the subject and comparing the measured level of TGFß-1 with a reference level of TGFß-1 from a healthy subject, and determining a level of TGFß-2 in a sample obtained from the subject and comparing the measured level of TGFß-2 with a reference level of TGFß-2 from a healthy subject, wherein:

an increased level of IL-23 compared with the reference level of IL-23, a decreased level of IL-10 compared with the reference level of IL-10, and a decreased level of TGFß-2 compared with a reference TGFß-2 level, and an increased level of TGFß-1 compared with a reference level of TGFß-1, is indicative of a probability of the inflammatory bowel disease being Crohn's disease as opposed to ulcerative colitis.

In one embodiment, the steps of detecting increased or decreased levels of the cytokines employs an immunoassay, typically a quantitative immunoassay, and ideally a sandwich immuno-assay or a lateral flow assay.

In one embodiment, the quantitative immunoassay is a quantitative ELISA.

In one embodiment, the method comprises the steps of contacting the blood sample derived from the subject with an assay device comprising a support and a first antibody that is specific for IL-10 and a second antibody that is specific for IL-23 (and optionally a third antibody that is specific for TGFß-1 and/or a fourth antibody that is specific for TGFß-2) anchored to the support, under conditions such that an immunospecific cytokine-antibody binding reaction can occur, and quantitatively detecting the presence of an immunospecific cytokine binding reaction.

The method may include the sequential steps of:

contacting the blood sample derived from the subject with the assay device;
contacting the assay device with a labelled secondary antibody (i.e. a biotin labelled detection antibody) configured to bind to cytokine captured on the device;
contacting the assay device with an imaging ligand configured to bind to the label (i.e. streptavidin-conjugated Cy3 equivalent dye), and
quantification of the cytokines captured on the device using an imaging quantification system (i.e. a laser fluorescent scanner system).

In one embodiment, the steps of detecting increased or decreased levels of the cytokines comprises quantitative detection of nucleic acids encoding the or each cytokine, for example quantitative real time PCR.

In another aspect, the invention provides a method of identifying a therapeutic regime for a subject with inflammatory bowel disease, comprising the steps of:
determining the probability of the inflammatory bowel disease in the subject being ulcerative colitis or Crohn's disease according to a method of the invention;
when a probability of the inflammatory disease being ulcerative colitis is indicated, identifying an ulcerative colitis therapeutic regime for the subject, or
when a probability of the inflammatory disease being Crohn's disease is indicated, identifying an ulcerative colitis therapeutic regime for the subject.

In another aspect, the invention provides a method of monitoring effectiveness of ulcerative colitis treatment regime in a subject with ulcerative colitis over a treatment period, comprising the steps of:
determining a level of IL-10 in a plurality of blood sample obtained from the subject at spaced apart time points during the treatment period; and
determining a level of IL-23 in a plurality of blood sample obtained from the subject at spaced apart time points during the treatment period,
   wherein a decrease in a level of IL-10 and an increase in a level of IL-23 during the treatment period is indicative of the ulcerative colitis treatment regime being effective, and wherein an increase or no change in a level of IL-10 and a decrease or no change in a level of IL-23 during the treatment period is indicative of the ulcerative colitis treatment regime being ineffective.

In one embodiment, the method includes the steps of:
determining a level of TGFß-1 in a plurality of blood sample obtained from the subject at spaced apart time points during the treatment period; and/or
determining a level of TGFß-2 in a plurality of blood sample obtained from the subject at spaced apart time points during the treatment period,
   wherein an increase in a level of TGFß-1 and/or a decrease in a level of TGFß-2 during the treatment period is indicative of the ulcerative colitis treatment regime being effective, and wherein a decrease or no change in a level of TGFß-1 and/or an increase or no change in a level of TGFß-2 during the treatment period is indicative of the ulcerative colitis treatment regime being ineffective.

In another aspect, the invention provides a method of monitoring effectiveness of a Crohn's disease treatment regime in a subject with Crohn's disease over a treatment period, comprising the steps of:
determining a level of IL-10 in a plurality of blood sample obtained from the subject at spaced apart time points during the treatment period; and
determining a level of IL-23 in a plurality of blood sample obtained from the subject at spaced apart time points during the treatment period,
   wherein an increase in a level of IL-10 and a decrease in a level of IL-23 during the treatment period is indicative of the Crohn's disease treatment regime being effective, and
   wherein a decrease or no change in a level of IL-10 and/or an increase or no change in a level of IL-23 during the treatment period is indicative of the Crohn's disease treatment regime being ineffective.

In one embodiment, the method includes the steps of:
determining a level of TGFß-1 in a plurality of blood samples obtained from the subject at spaced apart time points during the treatment period; and/or
determining a level of TGFß-2 in a plurality of blood sample obtained from the subject at spaced apart time points during the treatment period,
   wherein a decrease in a level of TGFß-1 and/or an increase in a level of TGFß-2 during the treatment period is indicative of the Crohn's disease treatment regime being effective, and wherein an increase or no change in a level of TGFß-1 and/or a decrease or no change in a level of TGFß-2 during the treatment period is indicative of the Crohn's disease treatment regime being ineffective.

In another aspect, the invention provides a method of treating an inflammatory bowel disease in a subject with inflammatory bowel disease, comprising the steps of determining the status of the inflammatory disease in the subject (i.e. determining a probability of the disease being one of ulcerative colitis or Crohn's disease, or diagnosis of one of ulcerative colitis or Crohn's disease) according to a method of the invention; and treating the disease according to the determined status. For example, when it is determined that the disease is ulcerative colitis, the subject is treated with an ulcerative colitis therapeutic regime. Likewise, when it is determined that the disease is Crohn's disease, the subject is treated with a Crohn's disease therapeutic regime.

The invention also provides a system for performing a method of the invention.

In one embodiment, the system comprises a blood analysis module configured to determine a level of IL-10 and IL23 in a blood sample, and a processing module configured to receive the determined levels of IL-10 and IL-23 from the blood analysis module, calculate a ratio of II-10 to IL-23, and provide an output of the likelihood of the patient having CD or UC based on the calculated ratio of IL-10 to IL-23. The processor may be configured to output a likelihood of the IBD being CD when the calculated IL-10 to IL-23 ratio is less than 1:1, for example less than 1:1.5, 1:2, or 1:2.5. The processor may be configured to output a likelihood of the IBD being UC when the calculated IL-10 to IL-23 ratio is greater than 1:1, for example greater than 1.5:1, 2:1 or 2.5:1.

In one embodiment, the system comprises a blood analysis module configured to determine a level of IL-10 and IL23 in a blood sample, and a processing module configured to receive the determined levels of IL-10 and IL-23 from the blood analysis module, compare the level of IL-10 with a reference IL-10 level and the level of IL-23 with a reference IL-23 level, and provide an output of the likelihood of the patient having CD or UC based on the comparison. The processor may be configured to output a likelihood of the IBD being UC when the measured IL-10 level is greater (for example at least a 1.5, 2.0 or 2.5 fold increase) than the reference IL-10 level and the measured IL-23 level is less (for example at least 0.1 or 0.15 fold decrease) than the reference IL-23 level. The processor may be configured to output a likelihood of the IBD being CD when the measured IL-10 level is less (for example at least a 0.1, 0.2 or 0.3 fold decrease) than the reference IL-10 level and the measured IL-23 level is greater (for example at least 0.2, 0.4 or 0.6 increase) than the reference IL-23 level.

Other aspects and preferred embodiments of the invention are defined and described in the other claims set out below.

### BRIEF DESCRIPTION OF THE FIGURES

FIG. 1: Fold change in IL-10 and IL-23 cytokines levels for patients with ulcerative colitis versus healthy subjects.

FIG. 2: Fold change in IL-10 and IL-23 cytokines levels for patients with Crohn's disease versus healthy subjects.

FIG. 3: Fold change in TGFß-1 and TGFß-2 cytokines levels for patients with Crohn's disease versus patients with ulcerative colitis versus healthy subjects.

FIG. 4: Fold change in IL-10, IL-23, TGFß-1 and TGFß-2 cytokine levels for patients with Crohn's disease versus patients with ulcerative colitis versus healthy subjects.

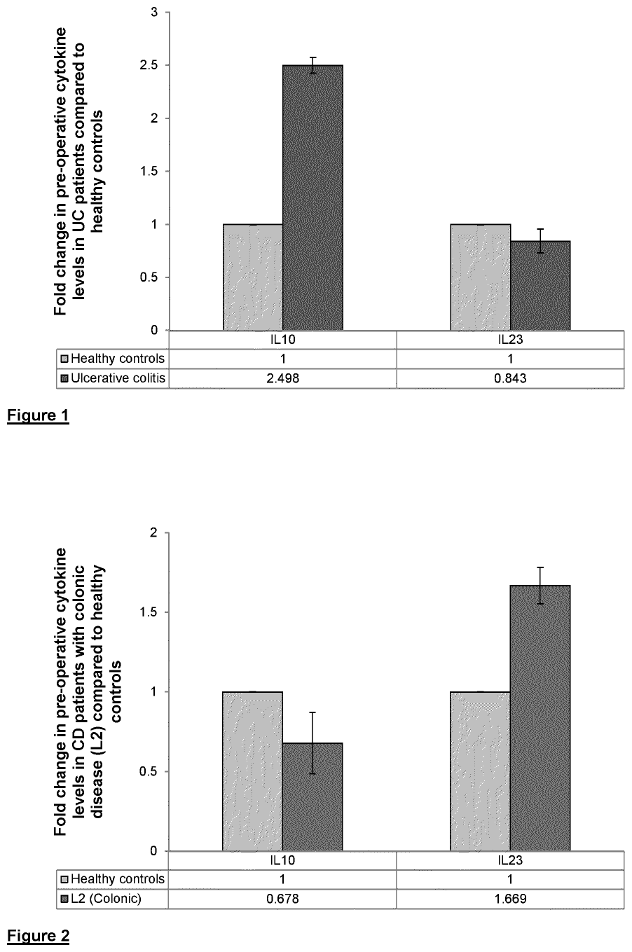

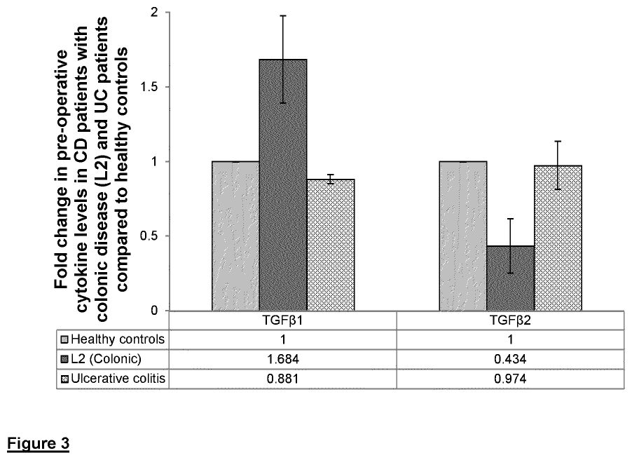

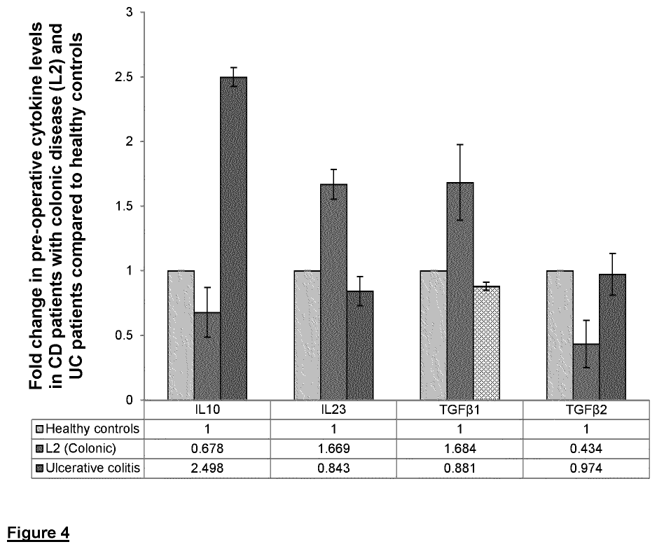

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure. Improvement may be observed in biological/molecular markers, clinical or observational improvements. In a preferred embodiment, the methods of the invention are applicable to humans, large racing animals (horses, camels, dogs), and domestic companion animals (cats and dogs).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra. The subject is generally symptomatic of IBD, as determined by art-recognised methods (i.e. loss of weight, abdominal cramps, blood in stool, blood or stool markers) and the method of the invention is employed to classify the IBD prior to diagnosis, prognosis or treatment of the disease. The patient may also have no symptoms, for example may be genetically predisposed to the disease due to ethnicity or family history, or be a smoker or be taking non-steroidal or anti-inflammatory medications.

The term “Inflammatory bowel disease” or “IBD” refers to a group of chronic, relapsing inflammatory conditions that are immunologically mediated. They include both Crohn's disease (CD) and ulcerative colitis (UC). In general, IBD is believed to result from an aggressive immunologic response in genetically susceptible individuals, usually due to an environmental factor, such as gut commensals. The methods of the invention are suitable for use with patients with IBD, and patients with symptoms of IBD. Symptoms include blood in the stool, abdominal cramps, loss of weight and apatite.

“Ulcerative colitis” or “UC” refers to a specific form of inflammatory bowel disease (IBD) that causes long-lasting inflammation and ulcers (sores) in your digestive tract. Ulcerative colitis affects the innermost lining of your large intestine (colon) and rectum. Symptoms usually develop over time, rather than suddenly. Ulcerative colitis symptoms can vary, depending on the severity of inflammation and where it occurs. Signs and symptoms may include: Diarrhea, often with blood or pus, Abdominal pain and cramping, Rectal pain, Rectal bleeding passing small amount of blood with stool, Urgency to defecate, Inability to defecate despite urgency, Weight loss, Fatigue, Fever, In children, failure to grow. The term “ulcerative colitis” includes different types of colitis, including Ulcerative proctitis, Proctosigmoiditis, Left-sided colitis, Pancolitis, and Acute severe ulcerative colitis.

“Ulcerative colitis therapeutic regime” refers to drug therapy and/or surgery. Drug therapy may involve an anti-inflammatory drug (i.e. 5-aminosalicyclates or corticosteroids), immune system suppressors (i.e. azathioprine or mercaptopurine, cyclosporine, Infliximab, Vedolizumab), antibiotics, anti-diarrheal drugs, pain medicaments and iron supplements. Surgery can often eliminate ulcerative colitis, and usually means removing the entire colon and rectum (proctocolectomy). In most cases, this involves a procedure called ileal pouch anal anastomosis. This procedure eliminates the need to wear a bag to collect stool. Your surgeon constructs a pouch from the end of your small intestine. The pouch is then attached directly to your anus, allowing you to expel waste relatively normally. In some cases a pouch is not possible. Instead, surgeons create a permanent opening in your abdomen (ileal stoma) through which stool is passed for collection in an attached bag.

“Crohn's disease” or “CD” is an inflammatory bowel disease (IBD). It causes inflammation of your digestive tract, which can lead to abdominal pain, severe diarrhea, fatigue, weight loss and malnutrition. Inflammation caused by Crohn's disease can involve different areas of the digestive tract in different people. In some people with Crohn's disease, only the last segment of the small intestine (ileum) is affected. In others, the disease is confined to the colon (part of the large intestine). The most common areas affected by Crohn's disease are the last part of the small intestine and the colon. The inflammation caused by Crohn's disease often spreads deep into the layers of affected bowel tissue. Crohn's disease can be both painful and debilitating, and sometimes may lead to life-threatening complications. While there's no known cure for Crohn's disease, therapies can greatly reduce its signs and symptoms and even bring about long-term remission. With treatment, many people with Crohn's disease are able to function well.

“Crohn's disease therapeutic regime” refers to refers to drug therapy, nutrition therapy and/or surgery. Drug therapy may involve an anti-inflammatory drug (i.e. oral 5-aminosalicyclates or corticosteroids), immune system suppressors (i.e. azathioprine or mercaptopurine, methotrexate, Infliximab, natalizumab, ustekinumab), antibiotics such as ciprofloxacin (Cipro) and metronidazole (Flagyl), anti-diarrheal drugs, pain medicaments, Vitamin B, calcium and vitamin D supplements and iron supplements. Nearly half of those with Crohn's disease will require at least one surgery. However, surgery does not cure Crohn's disease. Surgery generally involves removal of a damaged portion of your digestive tract and then reconnection with the healthy sections. Surgery may also be used to close fistulas and drain abscesses. Nutrition therapy may involve enteral or parenteral nutrition administration, or a low-fibre or low-residue diet. Enteral and parenteral nutrition are typically used to get people healthier prior to surgery or when other medications fail to control symptoms.

“Indeterminate Colitis”: distinguishing ulcerative colitis (UC) from Crohn's disease (CD) is normally based on evaluation of a variety of clinical, radiologic, serologic and pathologic findings, the latter in biopsy and/or resection specimens. Some patients with IBD show overlapping pathologic features of UC and CD, which makes definite distinction between these two disorders difficult or even impossible. In most instances of uncertainty, the patient shows clinical and pathologic features of UC, but in addition, the patient's colon resection specimen reveals one or more CD-like features. In this setting, a diagnosis of indeterminate colitis (IC) is given. In one aspect, the invention may be applied to determine the type of IBD in a patient diagnosed as being Indeterminate Colitis.

“Determining inflammatory bowel disease (IBD) status” in a subject broadly refers to one or more of the following: classifying the type of IBD, determining the probability of the disease being ulcerative colitis or Crohn's disease, distinguishing between ulcerative colitis or Crohn's disease, or detecting or predicting (risk of) ulcerative colitis or Crohn's disease. The subject is generally symptomatic of IBD, as determined by usual methods (i.e. loss of weight, abdominal cramps, blood in stool) and the method of the invention is employed to classify the IBD prior to treatment of the disease. The patient may also have no symptoms, but may be genetically predisposed to the disease.

“Determining the probability of inflammatory bowel disease in a subject being ulcerative colitis or Crohn's disease” broadly means determining whether the IBD in the subject is one or the other of the diseases (i.e. distinguish between UC and CD in a subject. This may be employed by a clinician to determine an appropriate treatment for the subject with (symptoms of) IBD. The cytokine profiles described herein may also be employed to detect or predict risk of UC or CD in a subject. In one embodiment, the subject with IBD has been diagnosed with Indeterminate Colitis (IC).

“IL-10”, also known as human cytokine synthesis inhibitory factor (CSIF), is an anti-inflammatory cytokine. In humans, interleukin 10 is encoded by the IL10 gene and signals through a receptor complex consisting of two IL-10 receptor-1 and two IL-10 receptor-2 proteins. Consequently, the functional receptor consists of four IL-10 receptor molecules. IL-10 binding induces STAT3 signalling via the phosphorylation of the cytoplasmic tails of IL-10 receptor 1+IL-10 receptor 2 by JAK1 and Tyk2 respectively. Assays for detection of IL-10, including quantitative detection, are known to a person skilled in the art, and include immunoassay based techniques (ELISA, lateral flow assays) and nucleic acid based techniques (real time PCR). Examples include IL-10 ELISA kits from Enzo Lifesciences, Invitrogen and Creative Diagnostics. IL-10 specific antibodies may be obtained from Abcam, Origene and Sigma-Aldrich. Nucleic acid-based assays for IL-10 are described in Piotnikova et al (J Immunol Methods. 2016 March; 430:51-5. doi: 10.1016/j.jim.2016.01.005. Epub 2016 Jan. 7).

“IL-23” or “Interleukin-23” is a heterodimeric cytokine composed of an IL12B (IL-12p40) subunit (that is shared with IL12) and the IL23A (IL-23p19) subunit (Immunity. 13 (5): 715-25). A functional receptor for IL-23 (the IL-23 receptor) has been identified and is composed of IL-12R β1 and IL-23R (Journal of Immunology. 168 (11): 5699-708). Assays for detection of IL-123, including quantitative detection, are known to a person skilled in the art, and include immunoassay based techniques (ELISA, lateral flow assays) and nucleic acid based techniques (real time PCR). Examples include IL-23 ELISA kits from Abcam, Thermofisher and MabTech. IL-23 specific antibodies are commercially available from RNDSystems, MabTech and EnzoLifeSciences. Nucleic acid-based assays for IL-23 are described in Liu et al (Rheumatology, Volume 46, Issue 8, August 2007, Pages 1266-1273), Kuchar et al (Proteins. 2014 June; 82(6): 975-989), Khodadadi et al (Iran J Med Sci. 2014 July; 39(4): 350-356) and Yang et al (Med Sci Monit. 2016; 22: 2785-2792). It will be appreciated that assays for IL-23 may comprise assays for a subunit of the cytokine, the details of which will be known to a person skilled in the art.

“TGFß-1” or “Transforming growth factor beta 1” is a polypeptide member of the transforming growth factor beta superfamily of cytokines. It is a secreted protein that performs many cellular functions, including the control of cell growth, cell proliferation, cell differentiation, and apoptosis. In humans, TGF-β1 is encoded by the TGFB1 gene. Assays for TGFß-1, including quantitative protein assays (i.e quantitative ELISA) or nucleic acid based assay (real time PCR), are well known to a person skilled in the art, and are commercially available from Biocompare.com, RNDsystmes.com, and Cellbiolabs.com. TGFß-1 specific antibodies are commercially available from Abcam.com, Origene.com and Biolegend.com.

“TGFß-2” or “Transforming growth factor-beta 2” is a secreted protein known as a cytokine that performs many cellular functions and has a vital role during embryonic development (alternative names: Glioblastoma-derived T-cell suppressor factor, G-TSF, BSC-1 cell growth inhibitor, Polyergin, Cetermin). It is an extracellular glycosylated protein. It is known to suppress the effects of interleukin dependent T-cell tumors. There are two named isoforms of this protein, created by alternative splicing of the same gene. (Int. J. Biochem. Cell Biol. 30 (3): 293-8). Assays for TGFß-1, including quantitative protein assays (i.e quantitative ELISA) or nucleic acid based assay (real time PCR), are well known to a person skilled in the art, and are commercially available from Biocompare.com, RNDsystmes.com, and Cellbiolabs.com. TGFß-1 specific antibodies are commercially available from Abcam.com, Origene.com and Biolegend.com.

“Biological sample” refers to any sample from the subject including blood, urine, stool, saliva, cerebrospinal fluid, tissue, and cells. Preferably, the sample is peripheral blood sample, or a fraction thereof.

“Blood sample” refers to blood, typically peripheral blood, or any fraction thereof, including plasma, serum or a cellular fraction from blood such as peripheral blood mononuclear cells (PBMC).

The term “reference level” as applied to a cytokine means a level of the cytokine in a healthy subject, i.e. a subject that does not have cancer or an inflammatory disease), and generally a level in peripheral blood of the subject. Methods of determining reference cytokine levels for subjects will be well known in the art. The methods of the invention involve detecting increased or decreased levels of the test cytokine in the subject compared with reference of the invention may involve detecting relative cytokine levels (where the subject sample is analysed in tandem with a sample from a reference subject), or absolute cytokine levels where specific abundance values are obtained and compared with a known reference level. In one embodiment, the method comprises detecting a fold change in the test cytokine value compared with a reference value. In this regard, increased levels of IL-10 generally include a fold change increase of at least 0.5 or 1.0. Likewise, decreased levels of IL-10 generally include a fold change decrease of at least 0.2 or 0.3. Increased levels of IL-23 generally include a fold change increase of at least 0.3 or 0.5. Likewise, decreased levels of IL-23 generally include a fold change decrease of at least 0.05 or 0.1. Increased levels of TGFß-1 generally include a fold change increase of at least 0.3 or 0.5. Likewise, decreased levels of TGFß-1 generally include a fold change decrease of at least 0.05 or 0.1. Decreased levels of TGFß-2 generally include a fold change decrease of at least 0.3 or 0.5. In one embodiment the method of the invention may be performed by comparing a ratio of IL-10 to IL-23, where a ratio of greater than 1:1 (for example a ratio of 1.2:1 or greater (for example 1.5:1 or greater, 2.0:1 or greater) is indicative of probability of the IBD being UC, and/or where a ratio of less than 1:1 (for example a ratio of 1:1.2 or less (for example 1:1.5 or less, 1:2 or less) is indicative of probability of the IBD being CD. In one embodiment, reference level as applied to the cytokines may be a reference level in a patient with confirmed CD or UC. Thus, if the ratio in the test levels of the cytokines (i.e. IL-10 and IL-23) matches that of a reference CD patient, or that of a reference UC patient, the patient may be determined to have a probability of the IBD being CD or UC, respectively.

“Immunoassay” or “quantitative immunoassay” as applied to a cytokine employed in the invention refers to a biochemical test that measures the concentration or a cytokine in a solution through the use of an antibody (usually) or an antigen (sometimes). The molecule detected by the immunoassay is often referred to as an “analyte” and is in many cases a protein, although it may be other kinds of molecules, of different size and types, as long as the proper antibodies that have the adequate properties for the assay are developed. Immunoassay types include ELISA, MELISA, CEDIA, multiplex arrays, magnetic immunoassays, radioimmunoassays, lateral flow tests, which are described in Yu et al (Scientific Reports volume 5, Article number: 11339 (2015)), Darwish et al (Int J Biomed Sci. 2006 September; 2(3): 217-235), Han et al (Annual Review of Analytical Chemistry, Vol. 6:119-141), Leng et al (J Gerontol A Biol Sci Med Sci).

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

The data presented in the figures was collected from patients who were all confirmed as UC or CD by endoscopy (pre-surgery) and histology (at time of endoscopic biopsy, and following surgical resection of tissue).

Crohn's disease patients, n=9

Ulcerative colitis patients, n=7

Self-declared healthy controls, n=4

Example of Cytokine Array Methods:
Human custom antibody array glass slides (Raybiotech Human Custom Antibody Array G Series—Raybiotech, Georgia, USA) were blocked for 30 minutes with blocking buffer (Raybiotech).
Slides were incubated, on a rocker, with whole plasma overnight at 4° C.
Slides were washed, on a rocker, a number of times using wash buffers (Raybiotech), at room temperature.
Slides were incubated, on a rocker, with biotinylated antibody cocktail (IL-10, IL-23, TGFß-1, TGFß-2) overnight at 4° C.
Slides were washed, on a rocker, a number of times using buffer (Raybiotech).
Slides were incubated, on a rocker, with IRDye 800CW Streptavidin (LI-COR Biosciences, United Kingdom) in the dark for 2 hours.
Slides were visualised on an Odyssey® SA ((LI-COR, United Kingdom).
Background signal was subtracted from values and data were normalised to the average of the positive controls within the replicate.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A method of treating a subject having an inflammatory bowel disease, comprising the steps of:
determining a status of the inflammatory bowel disease via a level of a plurality of biomarkers in a blood sample obtained from the subject with the inflammatory bowel disease, in which the biomarkers include IL-10 and IL-23; wherein
an increased level of IL-10 compared with a reference control level of IL-10 and a decreased level of IL-23 compared with a reference control level of IL-23 is indicative of a probability of the inflammatory bowel disease being ulcerative colitis as opposed to Crohn's disease; and
an increased level of IL-23 compared with the reference control level of IL-23 and a decreased level of IL-10 compared with the reference control level of IL-10 is indicative of a probability of the inflammatory bowel disease being Crohn's disease as opposed to ulcerative colitis; and
treating the inflammatory bowel disease according to the determined status.

2. A method according claim 1, in which the plurality of biomarkers consist of (a) IL-10, (b) IL-23, and (c) TGFß-1 and/or TGFß-2.

3. A method according claim 1, in which the plurality of biomarkers consist of IL-10 and IL-23.

4. A method according to claim 1, in which the subject has been diagnosed with indeterminate colitis.

5. A method according to claim 1, in which the subject has been diagnosed with indeterminate colitis by diagnostic analysis comprising a method selected from the group consisting of: scoping of the digestive tract; biopsy; MRI; CT scan; or ultrasound.

6. A method according to claim 1, including a step of determining a level of TGFß-1 in a sample obtained from the subject, wherein:
an increased level of IL-10 compared with a reference control level of IL-10, a decreased level of IL-23 compared with a reference control level of IL-23, and a decreased level of TGFß-1 compared with a reference control level of TGFß-1, is indicative of a probability of the inflammatory bowel disease being ulcerative colitis as opposed to Crohn's disease.

7. A method according to claim 1, including a step of determining a level of TGFß-1 in a sample obtained from the subject, wherein:
an increased level of IL-23 compared with a reference control level of IL-23, a decreased level of IL-10 compared with a reference control level of IL-10, and an increased level of TGFß-1 compared with a reference control level of TGFß-1, is indicative of a probability of the inflammatory bowel disease being Crohn's disease as opposed to ulcerative colitis.

8. A method according to claim 1, including a step of determining a level of TGFß-2 in a sample obtained from the subject, wherein:
an increased level of IL-10 compared with a reference control level of IL-10, a decreased level of IL-23 compared with a reference control level of IL-23, and an increased level of TGFß-2 compared with a reference control level of TGFß-2, is indicative of a probability of the inflammatory bowel disease being ulcerative colitis as opposed to Crohn's disease.

9. A method according to claim 1, including a step of determining a level of TGFß-2 in a sample obtained from the subject, wherein:
an increased level of IL-23 compared with a reference control level of IL-23, a decreased level of IL-10 compared with a reference control level of IL-10, and a decreased level of TGFß-2 compared with a reference control level of TGFß-2, is indicative of a probability of the inflammatory bowel disease being Crohn's disease as opposed to ulcerative colitis.

10. A method according to claim 1, including the steps of determining a level of TGFß-1 in a sample obtained from the subject, and determining a level of TGFß-2 in a sample obtained from the subject, wherein:
an increased level of IL-10 compared with the reference level of IL-10, a decreased level of IL-23 compared with the reference level of IL-23, a decreased level of TGFß-1 compared with a reference TGFß-1 level, and an increased level of TGFß-2 compared with a reference level of TGFß-2, is indicative of a probability of the inflammatory bowel disease being ulcerative colitis as opposed to Crohn's disease.

11. A method according to claim 1, including the steps of determining a level of TGFß-1 in a sample obtained from the subject, and determining a level of TGFß-2 in a sample obtained from the subject, wherein:
an increased level of IL-23 compared with the reference level of IL-23, a decreased level of IL-10 compared with the reference level of IL-10, and a decreased level of TGFß-2 compared with a reference TGFß-2 level, and an increased level of TGFß-1 compared with a reference level of TGFß-1, is indicative of a probability of the inflammatory bowel disease being Crohn's disease as opposed to ulcerative colitis.

12. A method according to claim 1, wherein the steps of detecting increased or decreased levels of the biomarkers employs a quantitative immuno-assay.

13-14. (canceled)

15. A method of treating a subject with ulcerative colitis over a treatment period, comprising the steps of:
determining a level of IL-10 in a plurality of blood samples obtained from the subject at spaced apart time points during the treatment period; and
determining a level of IL-23 in a plurality of blood samples obtained from the subject at spaced apart time points during the treatment period,
adjusting a treatment regime of the subject based on the levels of IL-10 and IL-23;
wherein a decrease in a level of IL-10 and an increase in a level of IL-23 during the treatment period is indicative of the ulcerative colitis treatment regime being effective, and wherein an increase or no change in a level of IL-10 and a decrease or no change in a level of IL-23 during the treatment period is indicative of the ulcerative colitis treatment regime being ineffective.

16. A method according to claim 15, including the steps of:
determining a level of TGFß-1 in a plurality of blood sample obtained from the subject at spaced apart time points during the treatment period; and/or
determining a level of TGFß-2 in a plurality of blood sample obtained from the subject at spaced apart time points during the treatment period,
wherein an increase in a level of TGFß-1 and/or a decrease in a level of TGFß-2 during the treatment period is indicative of the ulcerative colitis treatment regime being effective, and wherein a decrease or no change in a level of TGFß-1 and/or an increase or no change in a level of TGFß-2 during the treatment period is indicative of the ulcerative colitis treatment regime being ineffective.

17. A method of treating a subject with Crohn's disease over a treatment period, comprising the steps of:
determining a level of IL-10 in a plurality of blood samples obtained from the subject at spaced apart time points during the treatment period; and
determining a level of IL-23 in a plurality of blood samples obtained from the subject at spaced apart time points during the treatment period,
adjusting a treatment regime of the subject based on the levels of IL-10 and IL-23;
wherein an increase in a level of IL-10 and a decrease in a level of IL-23 during the treatment period is indicative of the Crohn's disease treatment regime being effective, and wherein a decrease or no change in a level of IL-10 and/or an increase or no change in a level of IL-23 during the treatment period is indicative of the Crohn's disease treatment regime being ineffective.

18. A method according to claim 17, including the steps of:
determining a level of TGFß-1 in a plurality of blood samples obtained from the subject at spaced apart time points during the treatment period; and/or
determining a level of TGFß-2 in a plurality of blood sample obtained from the subject at spaced apart time points during the treatment period,
wherein a decrease in a level of TGFß-1 and/or an increase in a level of TGFß-2 during the treatment period is indicative of the Crohn's disease treatment regime being effective, and wherein an increase or no change in a level of TGFß-1 and/or a decrease or no change in a level of TGFß-2 during the treatment period is indicative of the Crohn's disease treatment regime being ineffective.

19. A method according to claim 18, in which the subject has been diagnosed with Indeterminate Colitis (IC).
